(19) United States
(12) Patent Application Publication
(10) Pub. No.: US 2022/0009929 A1
(43) Pub. Date: Jan. 13, 2022

(54) POLYMORPHIC FORMS OF IBRUTINIB

(71) Applicant: CIPLA LIMITED, Mumbai, Maharashtra (IN)

(72) Inventors: Ramanaiah CHENNURU, Nellore(Dt), Andhra Pradesh (IN); Srinivas Laxminarayan PATHI, Bangalore, Karnataka (IN); Geena MALHOTRA, Mumbai (IN); Ramesh DEVARAPALLI, Krishna (Dist), Andhra Pradesh (IN)

(21) Appl. No.: 17/052,226

(22) PCT Filed: May 2, 2019
(86) PCT No.: PCT/IN2019/050349

§ 371 Date: Nov. 2, 2020

(30) Foreign Application Priority Data
May 2, 2018 (IN) 201821016611

(51) Int. Cl. C07D 487/04

(52) U.S. Cl. CPC C07D 487/04; C07B 2200/13

(57) ABSTRACT

Disclosed herein are crystalline polymorphic forms of Ibrutinib, methods for their preparation, pharmaceutical compositions comprising the polymorphic Forms, and their use thereof.

### FIELD OF INVENTION

The present invention relates to polymorphic forms of Ibrutinib, methods of preparation, pharmaceutical compositions comprising the polymorphic forms and their use for treating or preventing the activity of tyrosine kinase(s), thereof.

### BACKGROUND OF THE INVENTION

Ibrutinib of Formula I,

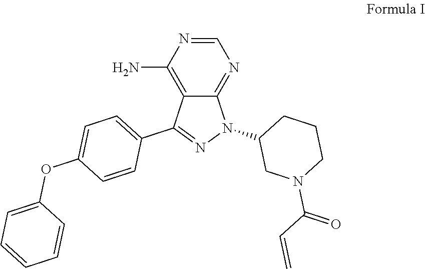

chemically named as 1-{(3R)-3-[4-Amino-3-(4-phenoxyphenyl)-1H-pyrazolo[3,4-d] pyrimidin-1-yl]-1-piperidinyl}-2-propen-1-one is an anticancer drug targeting B-cell malignancies. It is an orally-administered, selective and covalent inhibitor of the enzyme Bruton's tyrosine kinase (BTK).

International Patent Publication Number WO2013/184572 discloses crystalline forms of Ibrutinib A, B, C, D, E and F and also solvates. Also, discloses pharmaceutical compositions that include Ibrutinib, as well as methods of using it, alone or in combination with other therapeutic agents, for the treatment of autoimmune diseases or conditions, heteroimmune diseases or conditions, cancer, including lymphoma, and inflammatory diseases or conditions.

Application CN103694241 describes a new crystalline form of Ibrutinib, which is denoted therein as “Form A”.

International Patent Publication Number WO2015/145415 discloses crystalline Ibrutinib Forms III, IV, V, VI, VII, VIII and Form IX and processes for producing these crystalline forms. It also discloses stable amorphous Ibrutinib and processes for preparing stable amorphous Ibrutinib, pharmaceutical compositions comprising these forms and methods of using these crystalline and amorphous forms. Amorphous form of Ibrutinib is prepared by dry ball milling, wet ball milling, spray-drying techniques. The process also involves use of column chromatography to obtain amorphous form of Ibrutinib.

Regulatory agencies worldwide require a reasonable effort to identify the polymorphs of the drug substance and check for polymorph interconversions. Due to the often-unpredictable behaviour of polymorphs and their respective differences in physicochemical properties, consistency in manufacturing between batches of the same product must be demonstrated. Proper understanding of the polymorph landscape and nature of the polymorphs of a pharmaceutical will contribute to manufacturing consistency.

Thus, despite the existence of various processes for preparation of polymorphic forms of Ibrutinib, there remains a continuous need for new industrially feasible processes for the preparation of the same.

### OBJECTIVE OF THE INVENTION

The object of the present invention is to provide novel crystalline forms of Ibrutinib.

Another object of the present invention is to provide process for the preparation of crystalline forms of Ibrutinib.

Yet another object of the present invention is to provide pharmaceutical composition comprising a therapeutically effective amount of crystalline forms of Ibrutinib.

Yet another object of the present invention is to provide a process which is simple, economical and suitable for industrial scale-up.

### SUMMARY OF THE INVENTION

The present invention provides novel crystalline polymorphic forms of Ibrutinib.

The Ibrutinib may be a pseudo polymorphic form. Accordingly, pseudo polymorphs provided include hydrates and/or solvates.

In one embodiment, the present invention provides crystalline forms of Ibrutinib, hereinafter referred to as Form-C2 (1,2-dimethoxy ethane solvate), Form-C3, Form-C5 (benzyl alcohol solvate) and Form-C6 (acetic acid solvate).

The crystalline nature of forms according to the present invention is characterized by X-ray powder diffraction. Accordingly, the invention also provides methods for preparing these novel forms.

In another aspect, the present invention relates to processes for preparing novel polymorphic forms of Ibrutinib thereof.

The invention also provides pharmaceutical compositions comprising a therapeutically effective amount of at least one of the above described forms of Ibrutinib and at least one pharmaceutically acceptable excipient.

In yet another embodiment, the invention encompasses a process for preparing a pharmaceutical formulation comprising combining at least one of the above-described forms of Ibrutinib with at least one pharmaceutically acceptable excipient.

The invention also provides methods of treatment of diseases or symptoms wherein Ibrutinib is useful. In particular, these new methods are for similar therapeutic indications to those described in the above identified patents and applications and are incorporated herein by reference.

Further features of the present invention are defined in the dependent claims.

### BRIEF DESCRIPTION OF THE DRAWINGS

FIG. 1 represents a powder X-ray diffraction pattern of Form-C2 of Ibrutinib obtained according to example 1.

FIG. 2 represents a powder X-ray diffraction pattern of Form-C3 of Ibrutinib obtained according to example 2.

FIG. 3 represents a powder X-ray diffraction pattern of Form-C5 of Ibrutinib obtained according to example 4.

FIG. 4 represents a powder X-ray diffraction pattern of Form-C6 of Ibrutinib obtained according to example 5.

FIG. 5 represents a powder X-ray diffraction pattern of Form-C3 of Ibrutinib obtained according to example 6.

FIG. 6 represents a differential scanning calorimetry curve (DSC) of Form-C3 of Ibrutinib obtained according to example 6.

FIG. 7 represents a thermographic analysis (TGA) of Form-C3 of Ibrutinib obtained according to example 6.

### DETAILED DESCRIPTION OF THE INVENTION

Unless defined otherwise, technical and scientific terms used herein have the same meaning as commonly understood by one of ordinary skill in the art to which this invention belongs, and are consistent with:

As used herein, the term “XRPD” refers to powder X-ray diffraction, the term “IR” refers to infrared, the term “NMR” refers to nuclear magnetic resonance, the term “TGA” refers to thermogravimetric analysis, the term “DSC” refers to differential scanning calorimetry and the term “DVC” refers to dynamic vapour sorption isotherm.

As used herein, the term “substantially the same X-ray powder diffraction pattern” is understood to mean that those X-ray powder diffraction patterns having diffraction peaks with 20 values within +0.2° of the diffraction pattern referred to herein are within the scope of the referred to diffraction pattern.

As used herein, the term “solvate” refers to an association or complex of one or more solvent molecules and a compound of the invention. Such solvents for the invention may not interfere with the biological activity of the solute. Typically, the solvent used is a pharmaceutically acceptable solvent. Examples of solvents that form solvates include, but are not limited to, C1-C4 alcohol solvents such as isopropanol, ethanol, methanol, 1,2-dimethoxy ethane, benzyl alcohol, acetic acid, dimethyl sulfoxide (DMSO), 1,4-dioxane, tetrahydrofuran (THF), ethyl acetate and acetone, other than water at levels of more than 1%.

The solvate can be isolated either as an amorphous form or in a crystalline form, preferably in crystalline form.

The solvate can be further isolated either in anhydrous form or hydrated form.

As used herein, the term “hydrate” refers to the complex where the solvent molecule is water. The skilled person will appreciate that the water molecules are absorbed, adsorbed or contained within a crystal lattice of the solid compounds, usually in defined stoichiometric ratio. The notation for a hydrated compound may be nH₂O, where n is the number of water molecules per formula unit of the compound. For example, in a hemihydrate, n is 0.5; in a monohydrate n is one; in a sesquihydrate, n is 1.5; in a dihydrate, n is 2; and so on.

The novel polymorphs of the present invention may be isolated in pseudo polymorphic form as a solvate optionally in hydrated form, or as a non-hydrated solvate.

As polymorphic forms are reliably characterized by peak positions in the X-ray diffractogram, the polymorphs of the present invention have been characterized by powder X-ray diffraction spectroscopy which produces a fingerprint of the crystalline form and is able to distinguish it from all other crystalline and amorphous forms of Ibrutinib. Measurements of 20 values are accurate to within ±0.2 degrees. All the powder diffraction patterns were measured on a PANalytical X'Pert³ X-ray powder diffractometer with a copper-K-α radiation source.

As used herein the term “crystalline purity,” refers to a particular crystalline form of a compound in a sample which may contain amorphous form of the compound, one or more other crystalline forms of the compound other than the crystalline form of the compound of this invention, or a mixture thereof wherein the particular form of the compound is present in an amount of at least about 80%, preferably at least about 95%, most preferably at least about 99% crystalline.

The invention will now be described in detail in connection with certain preferred and optional embodiments, so that various aspects thereof may be more fully understood and appreciated.

The present invention provides novel crystalline forms of Ibrutinib designated as Form-C2, Form-C3, Form-C5 and Form-C6.

Thus, in one aspect, the present invention provides the crystalline Ibrutinib which is herein and in the claims designated as “Form-C2”, which has good flow characteristics.

In one embodiment, the crystalline Form-C2 is characterized by an X-ray powder diffraction pattern comprising the following 2θ values measured using CuKα, radiation.

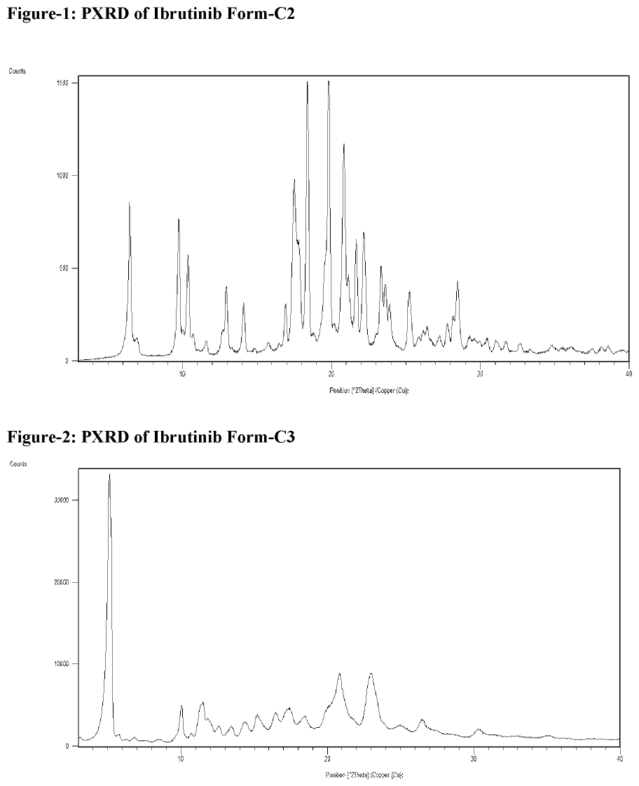

In an embodiment, the crystalline Form-C2 has an XRD pattern with characteristics peaks at 6.44, 9.72, 10.36, 16.92 and 18.37±0.2 °2θ. The XRPD diffractogram may comprise further peaks at 12.93, 14.09, 17.48, 19.79, 20.81, 21.64, 22.16, 25.22 and 28.44±0.2 °2θ. The XRPD diffractogram may be as depicted in FIG. 1.

The crystalline Form-C2 of Ibrutinib may be a 1,2-dimethoxy ethane solvate.

In an embodiment, the crystalline Form-C2 has an XRPD pattern with those peaks at ° 2θ values±0.2 °2θ as depicted in Table 1.

TABLE 1
Table of values for the XRPD pattern depicted in FIG. 1

| Peak value (°2θ) | Relative Intensity [%] |
|---|---|
| 6.44 | 46.8 |
| 9.72 | 49.63 |
| 10.36 | 35.46 |
| 12.93 | 24.68 |
| 14.09 | 18.02 |
| 16.92 | 6.45 |
| 17.48 | 59.7 |
| 18.37 | 100 |
| 19.79 | 98.72 |
| 20.81 | 68.76 |
| 21.64 | 38.96 |
| 22.16 | 42.19 |
| 25.22 | 22.57 |
| 28.44 | 25.33 |

Preferably the crystalline Form-C2 of Ibrutinib, has a crystalline purity of at least 80%, more preferably at least 90%, more preferably at least 95%, most preferably at least 99% by weight.

The invention encompasses a process for preparing the crystalline Form-C2 of Ibrutinib comprising, crystallization from 1,2-dimethoxy ethane. Crystallization may be accelerated by cooling the solution or seeding the solution with a crystal of Ibrutinib to obtain a precipitate; removing the resulting precipitate; and drying the solid.

Preferably, solution is cooled to a temperature of about −70° C. to about −60° C., preferably about −70° C.

Preferably, the solution is left without stirring for a period of about 1 hours to about 4 hours, preferably of about 2 hours to about 3 hours.

Preferably, the temperature of the reaction mixture is raised from about −70° C. to about 30° C.

Preferably, removing the precipitate is done by filtration.

Preferably, the obtained precipitate is dried to obtain a solid form.

Preferably, drying is at a temperature of about 20° C. to about 30° C., more preferably at about 25° C. Preferably, drying is performed for about 12 hours to about 24 hours, more preferably, for about 16 hours.

The invention also encompasses a crystalline form of Ibrutinib which is herein and in the claims designated as “Form-C3”, which has good flow characteristics.

In one embodiment, the crystalline Form-C3 is characterized by an X-ray powder diffraction pattern comprising the following 2θ values measured using CuKα, radiation.

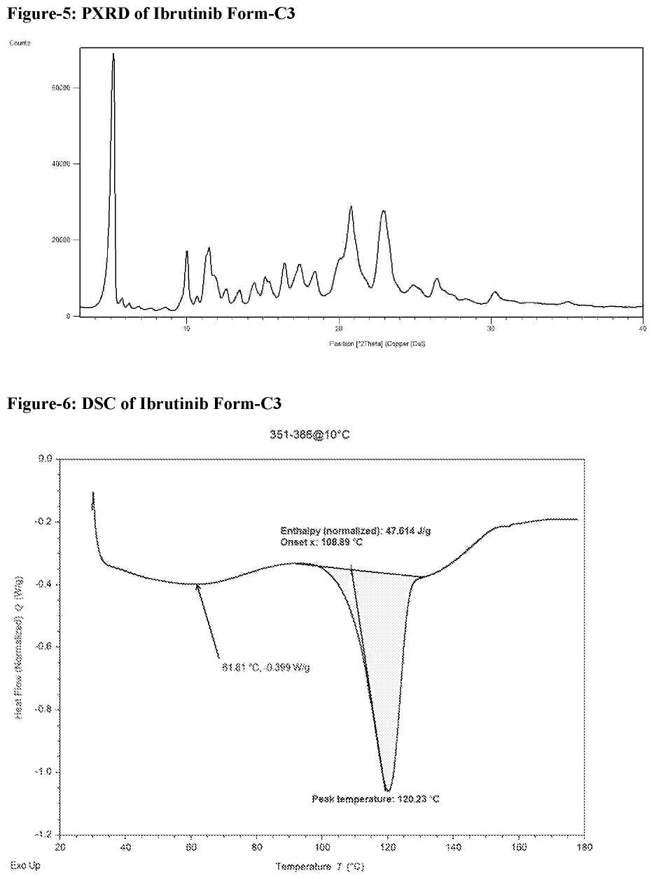

In an embodiment, the crystalline Form-C3 has an XRD pattern with characteristics peaks at 9.99, 15.26, and 17.32±0.2 °2θ. The XRPD diffractogram may comprise further peaks at 5.14, 11.51, 13.37, 14.35, 16.43, 18.40, 20.79, 22.99 and 26.47±0.2 °2θ. The XRPD diffractogram may be as depicted in the FIGS. 2 and 5.

In an embodiment, the crystalline Form-C3 has an XRPD pattern with those peaks at °2θ values±0.2 °2θ as depicted in Table 2.

TABLE 2
Table of values for the XRPD pattern depicted in FIGS. 2 and 5

| Peak value (°2θ) | Relative Intensity [%] |
|---|---|
| 5.14 | 100 |
| 9.99 | 12.75 |
| 11.51 | 6.67 |
| 13.37 | 4.09 |
| 14.35 | 5.89 |
| 15.26 | 7.73 |
| 16.43 | 7.79 |
| 17.32 | 10.42 |
| 18.40 | 6.22 |
| 20.79 | 23.33 |
| 22.99 | 18.23 |
| 26.47 | 6.32 |

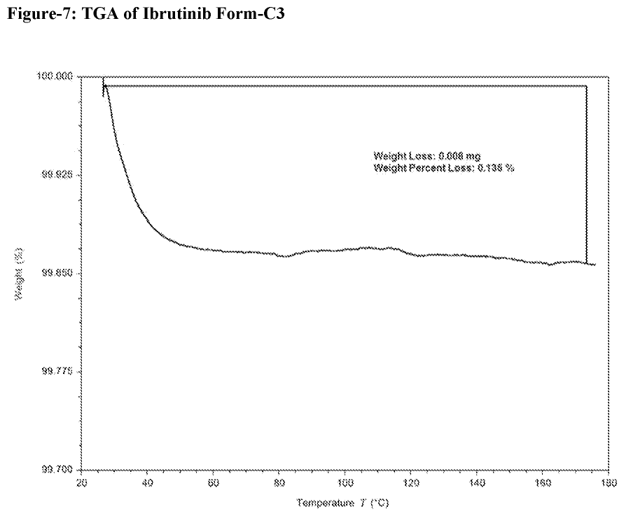

The crystalline nature of Form-C3 of Ibrutinib is further characterised by a DSC thermogram as depicted in FIG. 6. The DSC analysis was conducted using the Thermal Advantage (TA) Instruments 2500 DSC equipped with a refrigerated cooling system. DSC analysis indicated a single endothermic event was present at 120±3° C.

The crystalline nature of Form-C3 of Ibrutinib is further characterised by a thermal analysis (TGA) as depicted in FIG. 7. The TGA was conducted using Thermal Advantage (TA) Instruments Discovery 5500 series machine. FIG. 7 shows that thermal analysis (TGA) of Form-C3 indicated little or no weight loss up to 200° C. A small weight loss, typically less than 0.2%, was observed between 120 and 170° C., probably associated with inclusion of the crystallizing solvent in the crystals.

Thus, the crystalline nature of Form-C3 of Ibrutinib is characterised by the XRPD diffractogram as depicted in FIGS. 2 and 5; a DSC thermogram as depicted in FIG. 6; a TGA pattern as depicted in FIG. 7; and combinations thereof.

Preferably the crystalline Form-C3 of Ibrutinib, has a crystalline purity of at least 80%, more preferably at least 90%, more preferably at least 95%, most preferably at least 99% by weight.

The invention encompasses a process for preparing the crystalline Form-C3 of Ibrutinib comprising, crystallization from an alcohol. Crystallization may be accelerated by cooling the solution or seeding the solution with a crystal of Ibrutinib, to obtain a precipitate; stirring; removing the resulting precipitate; and drying the solid.

Preferably, alcohol is selected from methanol, ethanol, isopropanol, n-propanol, isobutanol, t-butanol, and the like. More preferably alcohol is methanol.

Preferably, stirring is about 1 min to about 60 mins, more preferably for about 1 min to about 30 mins at about 10° C. to about 40° C., more preferably at about 20° C. to about 30° C.

Preferably, the solution is left without stirring for a period of about 5 mins to about 30 mins, preferably of about 10 mins to about 20 mins, more preferably of about 10 mins to about 15 mins.

Preferably, removing the precipitate is done by filtration.

Preferably, the obtained precipitate is dried to obtain a solid form.

Preferably, drying is at a temperature of about 20° C. to about 30° C., more preferably at about 25° C. Preferably, drying is performed for about 12 hours to about 24 hours, more preferably, for about 16 hours.

Another process for preparing the crystalline Form-C3 of Ibrutinib comprises, crystallization from a mixture of an alcohol and water. Preferably, the crystallization comprises: dissolving Ibrutinib in alcohol; and adding water to the solution to obtain a precipitate.

Crystallization may be accelerated by cooling the solution or seeding the solution with a crystal of Ibrutinib, to obtain a precipitate; stirring; removing the resulting precipitate; and drying the solid.

Preferably, alcohol is selected from methanol, ethanol, isopropanol, n-propanol, isobutanol, t-butanol, and the like. More preferably alcohol is methanol.

Preferably, the amount of alcohol added is about 10 volumes to about 15 volumes per gram of Ibrutinib.

Preferably, reaction mass is heated to at about 40° C. to about 60° C., more preferably at about 45° C. to about 55° C. to get a clear solution.

Preferably, the reaction mass is clarified by the filtration, followed by a cooling step. Preferably, cooling is to about 20° C. to about 25° C.

Prior to the water addition, a seeding step may be performed. Preferably, seeding with Ibrutinib is done at about room temperature. Preferably, the seeding is done with the crystalline Form-C3 of Ibrutinib.

Following the seeding step, water is added. Preferably, the ratio of alcohol to water is about 1:1 to about 10:1, more preferably, about 3:1 to about 1.5:1.

Preferably, following water addition, a stirring step is performed. Preferably, stirring is for a period of about 10 minutes to about 60 minutes, preferably of about 20 minutes to about 50 minutes. Preferably, stirring is done at about 20° C. to about 25° C.

Preferably, removing the precipitate is done by filtration.

Preferably, the obtained precipitate is dried to obtain a solid form.

Preferably, drying is at a temperature of about 30° C. to about 80° C., more preferably at about 40° C. to about 60° C. Preferably, drying is performed for about 2 hours to about 12 hours, more preferably, for about 10 hours.

The invention also encompasses a crystalline form of Ibrutinib which is herein and in the claims designated as “Form-C5”, which has good flow characteristics.

In one embodiment, the crystalline Form-C5 is characterized by an X-ray powder diffraction pattern comprising the following 2θ values measured using CuKα, radiation.

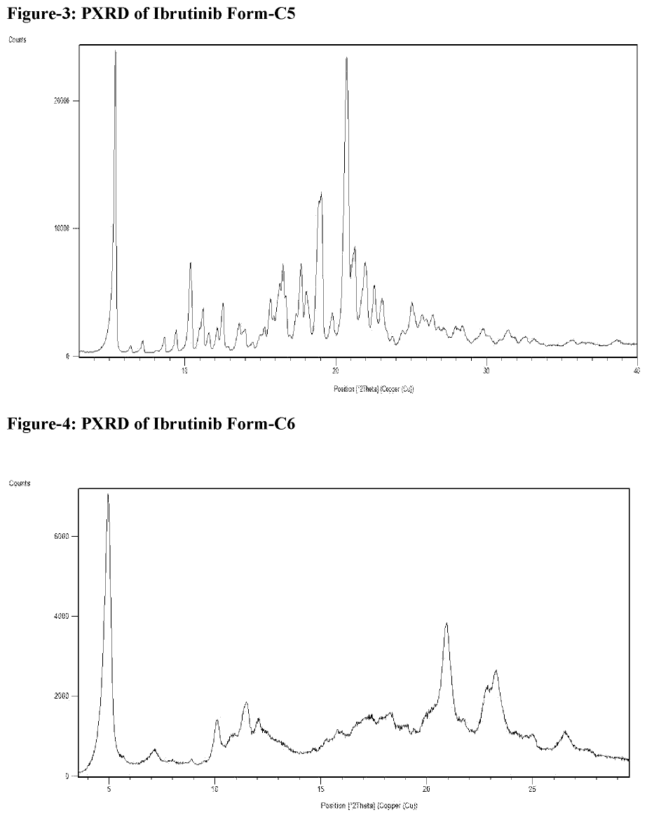

In an embodiment, the crystalline Form-C5 has an XRD pattern with characteristics peaks at 12.50, 17.66, and 22.54±0.2 °2θ. The XRPD diffractogram may comprise further peaks at 5.33, 7.17, 8.58, 9.38, 10.30, 11.17, 15.65, 16.47, 19.05, 20.66, 22.00 and 23.06±0.2 020. The XRPD diffractogram may be as depicted in FIG. 3.

The crystalline Form-C5 of Ibrutinib may be a benzyl alcohol solvate.

In an embodiment, the crystalline Form-C5 has an XRPD pattern with those peaks at ° 2θ values±0.2 °2θ as depicted in Table 3.

TABLE 3
Table of values for the XRPD pattern depicted in FIG. 3

| Peak value (°2θ) | Relative Intensity [%] |
|---|---|
| 5.33 | 50.14 |
| 7.17 | 2.36 |
| 8.58 | 2.72 |
| 9.38 | 4.07 |
| 10.30 | 12.74 |
| 11.17 | 9.18 |
| 12.50 | 8.71 |
| 15.65 | 8.71 |
| 16.47 | 10.54 |
| 17.66 | 15.39 |
| 19.05 | 23.77 |
| 20.66 | 39.06 |
| 22.00 | 12.9 |
| 22.54 | 9.82 |
| 23.06 | 8.98 |

Preferably the crystalline Form-C5 of Ibrutinib, has a crystalline purity of at least 80%, more preferably at least 90%, more preferably at least 95%, most preferably at least 99% by weight.

The invention encompasses a process for preparing the crystalline Form-C5 of Ibrutinib comprising, crystallising Ibrutinib from benzyl alcohol; stirring; removing the resulting precipitate; and drying the solid. Crystallization may be accelerated by cooling the solution, adding an antisolvent or seeding the solution with a crystal of Ibrutinib.

Preferred anti-solvents are n-heptane, n-hexane, MTBE, diisopropyl ether, diethyl ether or mixture thereof. Preferred anti-solvent combinations are MTBE/n-heptane and MTBE/n-hexane.

Preferably, after the antisolvent addition, a stirring step is performed for a period of about 1 hour to about 24 hours, preferably of about 2 hours to about 24 hours.

Preferably, stirring is done at a temperature of about 5° C. to about 30° C., more preferably, at a temperature of about 10° C. to about 30° C.

Preferably, removing the precipitate is done by filtration.

Preferably, the obtained precipitate is dried to obtain a solid form.

Preferably, drying is at a temperature of about 20° C. to about 30° C., more preferably at about 25° C. Preferably, drying is performed for about 12 hours to about 24 hours, more preferably, for about 16 hours.

The invention also encompasses a crystalline form of Ibrutinib which is herein and in the claims designated as “Form-C6”, which has good flow characteristics.

In one embodiment, the crystalline Form-C6 is characterized by an X-ray powder diffraction pattern comprising the following 2θ values measured using CuKα, radiation.

In an embodiment, the crystalline Form-C6 has an XRD pattern with characteristics peaks at 4.91, 11.42, and 23.23±0.2 °2θ. The XRPD diffractogram may comprise further peaks at 7.09, 10.09, 12.15, 20.90, 22.72 and 26.49±0.2° 2θ. The XRPD diffractogram may be as depicted in FIG. 4.

The crystalline Form-C6 of Ibrutinib may be an acetic acid solvate.

In an embodiment, the crystalline Form-C6 has an XRPD pattern with those peaks at °2θ values±0.2 °2θ as depicted in Table 4.

TABLE 4
Table of values for the XRPD pattern depicted in FIG. 4

| Peak value (°2θ) | Relative Intensity [%] |
|---|---|
| 4.91 | 100 |
| 7.09 | 4.99 |
| 10.09 | 13.78 |
| 11.42 | 15.21 |
| 12.15 | 9.88 |
| 20.90 | 44.27 |
| 22.72 | 12.81 |
| 23.23 | 28.95 |
| 26.49 | 6.83 |

Preferably the crystalline Form-C6 of Ibrutinib, has a crystalline purity of at least 80%, more preferably at least 90%, more preferably at least 95%, most preferably at least 99% by weight.

The invention encompasses a process for preparing the crystalline Form-C6 of Ibrutinib comprising, crystallising Ibrutinib from acetic acid; stirring; removing the resulting precipitate; and drying the solid. Crystallization may be accelerated by cooling the solution, adding an antisolvent or seeding the solution with a crystal of Ibrutinib.

The invention encompasses a process for preparing the crystalline Form-C6 of Ibrutinib by crystallizing it from a mixture of acetic acid and water. Preferably, the crystallization comprises: dissolving Ibrutinib in acetic acid; and adding water to the solution to obtain a precipitate

Preferably, the ratio of water to acetic acid is about 1:1 to about 10:1.

Optionally, following the water addition, a stirring step may be performed. Preferably, stirring is at about room temperature. Preferably, stirring is for a period of about 1 hour to about 24 hours, preferably of about 2 hours to about 20 hours. Preferably, stirring is done at about room temperature.

Optionally, following the stirring, the reaction mixture is left without stirring for a period of about 1 hour to about 24 hours, preferably of about 2 hours to about 20 hours, more preferably of about 5 hours to about 15 hours. Preferably, the reaction mixture is allowed to stand at about room temperature.

Preferably, removing the precipitate is done by filtration.

Preferably, the obtained precipitate is dried to obtain a solid form.

Preferably, drying is at a temperature of about 25° C. to about 50° C., more preferably at about 25° C. to about 30° C. Preferably, drying is performed for about 30 minutes to about 24 hours, preferably of about 40 minutes to about 20 hours, more preferably of about 60 minutes to about 15 hours.

Those skilled in the art would recognize that crystalline Forms C2, C3, C5 and C6 of the present invention may be further characterized by other methods including, but not limited to DSC, TGA, IR, solid state NMR, Raman spectroscopy, particle size, bulk density, tapped density, flow characteristic, solubility and intrinsic dissolution.

Ibrutinib, used in the preparation of crystalline forms C2, C3, C5 and C6 may be in any polymorphic form or in a mixture of any polymorphic forms.

Ibrutinib, used for the above process, as well as for the following processes, described in this application can be obtained by any method known to a skilled artisan.

The process of invention may be used as a method for purifying any form of ibrutinib, as well as for the preparation of the new polymorphic forms.

According to another aspect of the present invention, there is provided a pharmaceutical composition comprising polymorphic forms of ibrutinib as described above, together with one or more pharmaceutically acceptable carrier, diluent, vehicle or excipients. Ibrutinib, used in the preparation of pharmaceutical compositions may substantially consist of one of forms C2, C3, C5, or C6 as described above, or may substantially consist of a combination of two or more of said forms.

According to yet another aspect of the present invention there is provided use of polymorphic Forms C2, C3, C5 and C6 of ibrutinib as described above, in the preparation of a medicament useful in treating or preventing the activity of tyrosine kinase(s), such as Btk, or of treating a disease, disorder, or condition, which would benefit from inhibition of tyrosine kinase (s), such as Btk, in a mammal.

The invention is further defined by reference to the following examples describing in detail the preparation of ibrutinib crystalline forms of the invention. It will be apparent to those skilled in the art that many modifications, both to materials and methods, may be practiced without departing from the scope of the invention.

### EXAMPLES

### Example 1: Process to Prepare Form-C2 (1,2-Dimethoxy Ethane Solvate)

Ibrutinib (0.5 g) was dissolved in 1,2-dimethoxy ethane (2.5 ml) at Room temperature. The resulting solution was cooled to about −70° C. and left without stirring for 2 hrs to reach room temperature. The solids were isolated by filtration and dried under vacuum at 27° C. for 1 hr to result in the title compound. The solids were characterized by XRD as crystalline Form-C2, as presented in FIG. 1.

### Example 2: Process to Prepare Form-C3

Ibrutinib (2 g) was dissolved in methanol (4 ml) at room temperature, the solution shaked for 3 to 4 min and allowed to stand for 10 min. The solids were isolated by filtration and dried under vacuum at 27° C. for 1 hr to result in the title compound. The solids were characterized by XRD as crystalline Form-C3, as presented in FIG. 2.

### Example 3: Process to Prepare Form-C3

Ibrutinib (10 g) was dissolved in methanol (20 ml) at room temperature and stirred for 10 min. To the resulting slurry was added methanol (10 ml). The solids were isolated by filtration and dried under vacuum at 50° C. for 1 hr to result in the title compound. The solids were characterized by XRD as crystalline Form-C3, as presented in FIG. 2.

### Example 4: Process to Prepare Form-C5 (Benzyl Alcohol Solvate)

Ibrutinib (5 g) was dissolved in benzyl alcohol (10 ml) at room temperature. To this solution, 50 ml of MTBE and 100 ml of n-heptane were added and the resulting solution was stirred at room temperature for 3 hrs. The solids were characterized by XRD as crystalline Form-C5, as presented in FIG. 3.

### Example 5: Process to Prepare Form-C6 (Acetic Acid Solvate)

Ibrutinib (5 g) was dissolved in acetic acid (10 ml) at room temperature. To this solution, water (100 ml) was added and the resulting solution was stirred at room temperature for 8 hrs. The resulting slurry was allowed to stand at room temperature for 9 hrs. The solids were isolated by filtration and dried under vacuum at 27° C. for 1 hr to result in the title compound. The solids were characterized by XRD as crystalline Form-C6, as presented in FIG. 4.

### Example 6: Process to Prepare Form-C3

Ibrutinib (200 g) was dissolved in methanol (3.0 lit) at 50-55° C. and stirred for 20 min. The reaction mass was filtered and the clear filtrate was cooled to 23-25° C. The resulting filtrate was optionally seeded by addition of Form-C3 (0.5 g). The resulting reaction mass was diluted with water (2.0 lit). The resulting slurry was further stirred for 30 minutes at 23-25° C. The solids were isolated by filtration and dried under vacuum at 30° C. for 2 hrs and then at 45-50° C. for 6 hrs to result in the title compound. The solids were characterized as crystalline Form-C3 by XRD as presented in FIG. 5; by DSC as presented in FIG. 6 and by TGA as presented in FIG. 7.

1. A crystalline polymorphic form of Ibrutinib, selected from:
(a) Form-C2, which is characterized by having an XRPD diffractogram comprising peaks at 6.4, 9.7, 10.4, 16.9, and 18.4±0.2 °2Θ;
(b) Form-C3, which is characterized by having an XRPD diffractogram comprising peaks at 10.0, 15.3, and 17.3±0.2 °2Θ;
(c) Form-C5 which is characterized by having an XRPD diffractogram comprising peaks at 12.5, 17 and 22.5±0.2 °2Θ; and;
(d) Form-C6 which is characterized by having an XRPD diffractogram comprising peaks at 4.9, 11.4, and 23.2±0.2 °2Θ.

2. The crystalline polymorphic form of claim 1, wherein the form is Form-C3 of Ibrutinib and wherein the Form-C3 of Ibrutinib is characterized by having an XRPD diffractogram as depicted in FIG. 2 or 5.

3. The crystalline polymorphic form of claim 1, wherein the form is Form-C3 of Ibrutinib, wherein the Form-C3 of Ibrutinib is further characterized by having an XRPD diffractogram comprising peaks at, 5.1, 11.5, 13.4, 14.4 16.4, 18.4, 20.8, 23.0, and 26.5±0.2 °2Θ.

4-7. (canceled)

8. The crystalline polymorphic form of claim 1, wherein the form is Form-C3 of Ibrutinib, further characterized by DSC thermogram having an endothermic peak single endothermic event present at 120±3° C., a weight loss when heating to a temperature of about 100° C. of less than about 2% as measured by TGA; or a combination thereof.

9. A process for preparing crystalline Form-C3 of Ibrutinib, as claimed in claim 1, wherein, the process comprises dissolution of ibrutinib in an alcohol solvent to form a solution, and crystallization from the solution.

10. The process according to the claim 9 wherein the dissolution is performed by stirring the alcohol solvent.

11. The process according to the claim 10, wherein the alcohol is methanol.

12. The process according to claim 10, wherein stirring is conducted for about 1 min to about 60 mins.

13. The process according to the claim 12, wherein after stirring the solution is left without stirring for a period of about 5 mins to about 30 mins to yield a precipitated product.

14-16. (canceled)

17. The process according to the claim 9 further comprising adding water to the alcohol solution.

18. (canceled)

19. The process according to the claim 17, wherein the alcohol is methanol.

20. The process according to the claim 9, wherein 10-15 ml of alcohol added is per gram of Ibrutinib.

21. The process according to the claim 9, wherein the alcohol solvent is heated to at about 40° C. to about 60° C. to obtain a clear solution.

22. The process according to the claim 21, wherein the alcohol solvent is clarified by the filtration, followed by cooling to about 20° C. to about 25° C.

23. The process according to the claim 22, wherein the clarification is performed prior to the water addition, further comprising seeding the alcohol solution with crystalline Form-C3 of Ibrutinib.

24. The process according to the claim 22, wherein water is added after the clarification step at about 20° C. to about 25° C.

25. The process according to the claim 17 wherein, the ratio of alcohol to water is about 1:1 to about 10:1.

26-28. (canceled)

29. Crystalline Form-C3 of Ibrutinib prepared by a process according to claim 9.

30. A pharmaceutical composition comprising: (a) a therapeutically effective amount of a crystalline Form-C3 of Ibrutinib according to claim 1; and (b) at least one pharmaceutically acceptable carrier, diluent, vehicle or excipient.

31. A method for treating or preventing the activity of tyrosine kinase(s), such as Btk, or of treating a disease, disorder, or condition, which benefits from inhibition of tyrosine kinase(s), such as Btk, in a mammal, the method comprises administering therapeutically effective amounts to a patient in need thereof crystalline Form-C3 of Ibrutinib according to claim 1.
